(19) United States
(12) Patent Application Publication
(10) Pub. No.: US 2026/0129310 A1
(43) Pub. Date: May 7, 2026

(54) LIVING BODY INFORMATION ACQUISITION APPARATUS AND NON-TRANSITORY COMPUTER-READABLE STORAGE MEDIUM STORING LIVING BODY INFORMATION ACQUISITION PROGRAM

(71) Applicant: SEIKO EPSON CORPORATION, Tokyo (JP)

(72) Inventors: Akira IKEDA, CHINO-SHI (JP); Satoshi SHIMIZU, SHIOJIRI-SHI (JP)

(73) Assignee: SEIKO EPSON CORPORATION, Tokyo (JP)

(21) Appl. No.: 19/315,692

(22) Filed: Sep. 1, 2025

(30) Foreign Application Priority Data
Sep. 4, 2024 (JP) 2024-152088

(51) Int. Cl. H04N 23/84; A61B 5/021; H04N 25/11

(52) U.S. Cl. CPC H04N 23/84; H04N 25/11; A61B 5/02108

(57) ABSTRACT

A living body information acquisition apparatus includes: an imaging portion configured to capture reflected light reflected off a living body and containing first wavelength light having a first wavelength and second wavelength light having a second wavelength different from the first wavelength, and output a first output value relating to the first wavelength light and a second output value relating to the second wavelength light; a data processing portion configured to perform data correction on the first output value to cause the first output value to approach a median derived from an output resolution of the imaging portion; and a calculation portion configured to calculate living body information based on the first output value and the second output value.

The present application is based on, and claims priority from JP Application Serial Number 2024-152088, filed Sep. 4, 2024, the disclosure of which is hereby incorporated by reference herein in its entirety.

### BACKGROUND

### 1. Technical Field

The present disclosure relates to a living body information acquisition apparatus and a non-transitory computer-readable storage medium storing a living body information acquisition program.

### 2. Related Art

There is a known pulse wave measurement apparatus that acquires living body information such as a pulse wave. The pulse wave measurement apparatus is an example of the living body information acquisition apparatus. A pulse wave measurement apparatus described in JP-A-2021-183079 includes an imaging device, a display device, and an input device. The imaging device has three channels: a channel R (red); a channel G (green); and a channel B (blue). The pulse wave measurement apparatus measures a pulse wave signal from a living body by using video images of a face region of the living body captured by the imaging device.

JP-A-2021-183079 is an example of the related art.

Signals output from the three channels R, G, and B and carrying video images of the face region of a living body captured by the imaging device differ from each other. Since the signals output from the three channels R, G, and B differ from each other, the accuracy of detection of the pulse wave signal decreases in some cases.

### SUMMARY

A living body information acquisition apparatus according to an aspect of the present disclosure includes: an imaging portion configured to capture reflected light reflected off a living body and containing first wavelength light having a first wavelength and second wavelength light having a second wavelength different from the first wavelength, and output a first output value relating to the first wavelength light and a second output value relating to the second wavelength light; a data processing portion configured to perform data correction on the first output value to cause the first output value to approach a median derived from an output resolution of the imaging portion; and a calculation portion configured to calculate living body information based on the first output value and the second output value.

A living body information acquisition apparatus according to another aspect of the present disclosure includes: a color filter configured to transmit reflected light reflected off a living body and containing first wavelength light having a first wavelength and second wavelength light having a second wavelength different from the first wavelength; an imaging portion configured to capture the reflected light passing through the color filter and output a first output value relating to the first wavelength light and a second output value relating to the second wavelength light; and a calculation portion configured to calculate living body information based on the first output value and the second output value, and the color filter has light transmittance that causes the first output value to approach a median derived from an output resolution of the imaging portion.

A non-transitory computer-readable storage medium storing a living body information acquisition program according to another aspect of the present disclosure is configured to cause a computer coupled to an imaging unit including an imaging portion configured to capture reflected light reflected off a living body and containing first wavelength light having a first wavelength and second wavelength light having a second wavelength different from the first wavelength and output a first output value relating to the first wavelength light and a second output value relating to the second wavelength light to calculate living body information based on the first output value and the second output value on which data correction that causes the first output value and the second output value to approach a median derived from an output resolution of the imaging portion is performed.

### BRIEF DESCRIPTION OF THE DRAWINGS

FIG. 1 shows a schematic configuration of a measurement apparatus.

FIG. 2 shows a schematic configuration of an imaging unit.

FIG. 3 is a block diagram showing the configuration of the measurement apparatus.

FIG. 4 shows an example of signal values.

FIG. 5 shows an example of the signal values.

FIG. 6 shows an example of grayscale values.

FIG. 7 shows an example of the grayscale values.

FIG. 8 shows an example of the signal values.

FIG. 9 shows an example of the signal values.

FIG. 10 shows an example of the signal values.

FIG. 11 shows an example of a detection region.

FIG. 12 shows an example of detected grayscale values.

FIG. 13 shows an example of an analysis procedure for detecting a pulse wave signal.

FIG. 14 shows a result of analysis of a noise-removed signal.

FIG. 15 shows an example of a control procedure executed by the measuring apparatus.

### DESCRIPTION OF EMBODIMENTS

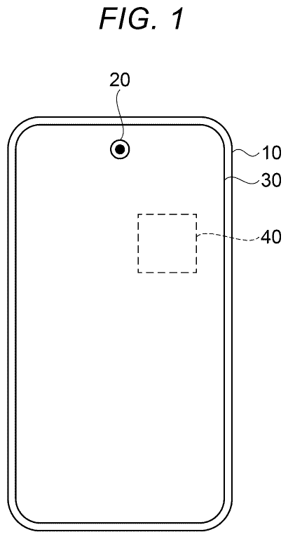

FIG. 1 shows a schematic configuration of a measurement apparatus 10. The measurement apparatus 10 corresponds to an example of the living body information measurement apparatus. The measurement apparatus 10 detects a pulse wave signal from a measurement operator by using video image data. The pulse wave signal is a signal indicating the pulse wave of the measurement operator, and is an example of the living body information. The measurement operator corresponds to an example of the living body. The measurement apparatus 10 calculates the living body information such as the pulse, pulse fluctuation, oxygen saturation concentration, and blood pressure based on the pulse wave signal. The measurement apparatus 10 may evaluate sleep apnea syndrome or the like based on the living body information. The measurement apparatus 10 displays the living body information including the pulse wave signal.

The measurement apparatus 10 is configured with an information processing apparatus. The measurement apparatus 10 shown in FIG. 1 is a smartphone, but is not limited thereto. The measurement apparatus 10 only needs to be an apparatus having the function of capturing video images or an apparatus couplable to a device that captures video images. The measurement apparatus 10 is configured with a desktop computer, a laptop computer, a tablet terminal, or the like. The measurement apparatus 10 includes an imaging unit 20, a display unit 30, and a control unit 40.

The imaging unit 20 captures reflected light L reflected off the measurement operator. The reflected light L contains outside light. The imaging unit 20 generates video image data containing the face of the measurement operator by capturing the reflected light L. The video image data is configured with multiple sets of image data. The multiple sets of image data are each configured with multiple signal values. The image data is data used to cause the display unit 30 to display a captured image 100. The captured image 100 is a still image. The imaging unit 20 captures images at a predetermined frame rate to generate the video image data.

The imaging unit 20 shown in FIG. 1 is a smartphone camera built in the measurement apparatus 10. The imaging unit 20 may be an external camera externally attached to the measurement apparatus 10. The external camera is a near-infrared camera, a web camera, or the like. The imaging unit 20 may include a lens unit, an optical filter, and the like externally attached to the built-in smartphone camera.

The display unit 30 displays various pieces of information such as the captured image 100. The display unit 30 displays various pieces of living body information including the pulse wave signal. The display unit 30 may display a comment or the like based on the living body information. The display unit 30 is configured with a liquid crystal panel, an organic electro-luminescence (EL) panel, or the like.

The display unit 30 has a touch input function. The display unit 30 functions as an input unit. The display unit 30 accepts various input operations performed by the measurement operator. The display unit 30 generates various input signals according to the input operations. The display unit 30 shown in FIG. 1 has the touch input function, but not necessarily. An externally attached mouse, keyboard, touch panel, or the like may be used as an input device of the measurement apparatus 10.

The control unit 40 controls various units such as the imaging unit 20 and the display unit 30. The control unit 40 controls the various units to cause them to measure the living body information including the pulse wave signal. The control unit 40 may control the various units to cause the measurement apparatus 10 to perform an operation other than the measurement of the living body information.

The measurement operator operates the measurement apparatus 10 at a position where the measurement operator faces the imaging unit 20 of the measurement apparatus 10. The measurement operator operates the measurement apparatus 10 when causing the measurement apparatus 10 to detect the living body information. The measurement operator may operate the measurement apparatus 10 when performing a task such as document creation. The measurement apparatus 10 detects the living body information relating to the measurement operator in the background when the measurement operator is performing a task such as document creation. The measurement apparatus 10 can detect the living body information relating to the measurement operator in a typical active state by detecting the living body information in the background.

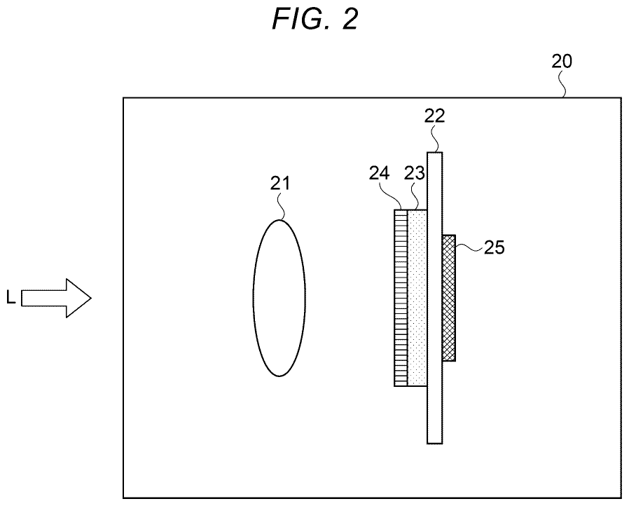

FIG. 2 shows a schematic configuration of the imaging unit 20. The imaging unit 20 includes an optical element 21, a substrate 22, an imaging sensor 23, a color filter 24, and an image processing circuit group 25.

The optical element 21 brings the reflected light L into focus at the imaging sensor 23. The optical element 21 is configured with one or more lenses, mirrors, diffraction gratings, and the like. The configuration of the optical element 21 is not limited to a specific configuration as long as the optical element 21 is configured to bring the reflected light L into focus at the imaging sensor 23.

The substrate 22 supports the imaging sensor 23 and the image processing circuit group 25. The substrate 22 may support a support frame that is not shown, and the support frame supports the optical element 21. The substrate 22 supports the optical element 21 via the support frame.

The imaging sensor 23 converts the luminance of the reflected light L brought into focus by the optical element 21 into an electric signal. The imaging sensor 23 is configured with a charge coupled device (CCD), a complementary metal oxide semiconductor (CMOS) device, or the like. The imaging sensor 23 includes multiple pixels. The imaging sensor 23 converts the luminance of the reflected light L into an electric signal for each of the pixels.

The color filter 24 selectively transmits light having a predetermined wavelength. The color filter 24 selectively transmits red light RL, green light GL, and blue light BL by way of example. The red light RL corresponds to an example of first wavelength light. The green light GL corresponds to an example of second wavelength light. The blue light BL corresponds to an example of third wavelength light. The red light RL, the green light GL, and the blue light BL have different wavelengths. A red wavelength that is the wavelength of the red light RL ranges from 600 nm to 800 nm. The red wavelength corresponds to an example of a first wavelength. A green wavelength that is the wavelength of the green light GL ranges from 520 nm to 550 nm. The green wavelength corresponds to an example of a second wavelength. A blue wavelength that is the wavelength of the blue light BL ranges from 430 nm to 490 nm. The blue wavelength corresponds to an example of a third wavelength.

The color filter 24 has a first portion that transmits the red light RL, a second portion that transmits the green light GL, and a third portion that transmits the blue light BL. The first, second, and third portions are provided at positions where the three portions each face the corresponding pixels. The color filter 24 separates the reflected light L in terms of color into the red light RL, the green light GL, and the blue light BL. The color filter 24 is, for example, a Bayer filter. The color filter 24 may have, for example, a portion that transmits a near-infrared light.

The multiple pixels provided in the imaging sensor 23 capture the red light RL, the green light GL, and the blue light BL. The pixels facing the first portion each convert the red light RL into a red electric signal. The pixels facing the second portion each convert the green light GL into a green electric signal. The pixels facing the third portion each convert the blue light BL into a blue electric signal. The red, green, and blue electric signals are signals output on a pixel basis. The red, green, and blue electric signals are each an analog signal. The imaging sensor 23 outputs the electric signals to the image processing circuit group 25. The imaging sensor 23 corresponds to an example of an imaging portion.

The image processing circuit group 25 receives the red, green, and blue electric signals output from the imaging sensor 23, and performs various types of processing such as adjustment. The image processing circuit group 25 performs A/D conversion on each of the electric signals to convert the analog signal into a digital signal. The A/D conversion is an abbreviation for analog-to-digital conversion. The image processing circuit group 25 performs various types of digital signal processing on the digital signals as a result of the A/D conversion and outputs the processed signals to the control unit 40.

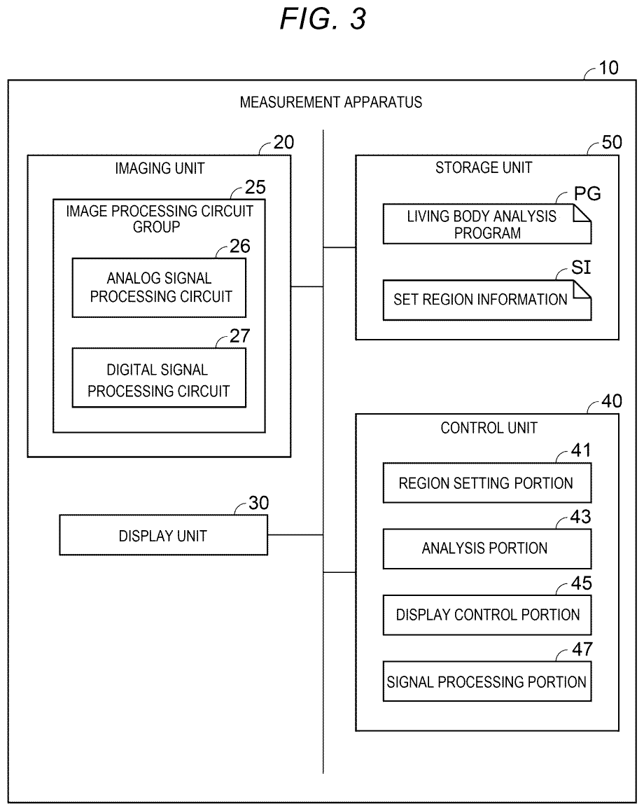

FIG. 3 is a block diagram showing the configuration of the measurement apparatus 10. The measurement apparatus 10 includes the imaging unit 20, the display unit 30, the control unit 40, and a storage unit 50. The measurement apparatus 10 may include a communication unit that is not shown, or the like. The storage unit 50 stores a living body analysis program PG and the like.

Based on a signal value of each of the electric signals, the imaging unit 20 generates a grayscale value corresponding to the signal value, and outputs video image data containing the grayscale values to the control unit 40. The grayscale values are each a digital signal and has a red grayscale value Gr relating to the red light RL, a green grayscale value Gg relating to the green light GL, and a blue grayscale value Gb relating to the blue light BL. The imaging unit 20 includes the image processing circuit group 25. The image processing circuit group 25 includes an analog signal processing circuit 26 and a digital signal processing circuit 27.

The analog signal processing circuit 26 receives each of the electric signals output from the imaging sensor 23, and adjusts the signal value, which is the magnitude of the electric signal. The analog signal processing circuit 26 receives a red signal value Sr, a green signal value Sg, and a blue signal value Sb as the signal values. The analog signal processing circuit 26 performs the adjustment on each of the signal values. Since the analog signal processing circuit 26 performs the adjustment on each of the signal values, the measurement apparatus 10 can improve the accuracy of the detection of the pulse wave signal. The adjustment corresponds to an example of data correction. As an example, the analog signal processing circuit 26 performs adjustment that adjusts a signal amplification factor for each of the signal values. The analog signal processing circuit 26 adjusts the signal amplification factor for each of the signal values to increase or decrease the signal value. The analog signal processing circuit 26 causes each of the signal values to approach an output median MV derived from the output resolution of the imaging sensor 23 by performing the adjustment. The analog signal processing circuit 26 corresponds to an example of a data processing portion.

The analog signal processing circuit 26 converts each of the signal values as a result of the adjustment into a grayscale value. The analog signal processing circuit 26 converts the red signal value Sr, the green signal value Sg, and the blue signal value Sb into the red grayscale value Gr, the green grayscale value Gg, and the blue grayscale value Gb, respectively. The analog signal processing circuit 26 outputs grayscale values including the red grayscale value Gr, the green grayscale value Gg, and the blue grayscale value Gb to the digital signal processing circuit 27.

The adjustment performed by the analog signal processing circuit 26 is, for example, color temperature adjustment using a color temperature. In the color temperature adjustment, the analog signal processing circuit 26 detects the color temperatures relating to the image data based on the electric signals. The imaging unit 20 acquires video image data in a preparation step before the measurement of the pulse wave signal. The imaging unit 20 detects the color temperatures relating to the image data contained in the video image data. The imaging unit 20 determines the correction coefficients to be used in the adjustment by using the detected color temperatures and a correction coefficient table. The correction coefficients are used to correct the magnitudes of the electric signals.

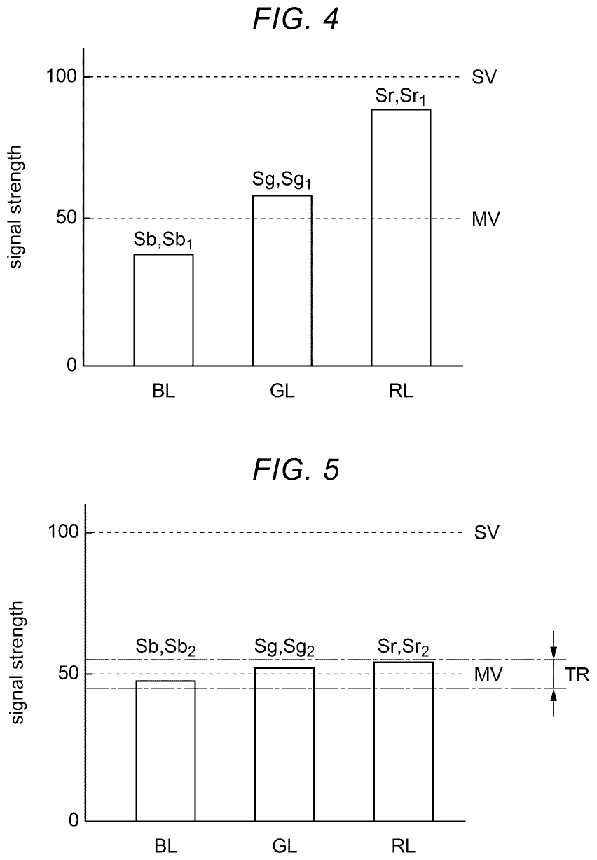

FIG. 4 shows an example of the signal values. FIG. 4 shows the red signal value Sr, the green signal value Sg, and the blue signal value Sb. The red signal value Sr indicates the magnitude of the electric signal relating to the red light RL. The red signal value Sr corresponds to an example of a first output value. The green signal value Sg indicates the magnitude of the electric signal relating to the green light GL. The green signal value Sg corresponds to an example of a second output value. The blue signal value Sb indicates the magnitude of the electric signal relating to the blue light BL. The blue signal value Sb corresponds to an example of a third output value.

FIG. 4 shows a saturated value SV and the output median MV characteristic of the imaging sensor 23. The saturated value SV is a value at which a signal value that is a result of the detection performed by the imaging sensor 23 is saturated. The output median MV corresponds to the median derived from the output resolution of the imaging sensor 23.

FIG. 4 shows a first red signal value Sr₁, a first green signal value Sg₁, and a first blue signal value Sb₁, which are the red signal value Sr, the green signal value Sg, and the blue signal value Sb before the adjustment is performed. The first red signal value Sr₁, the first green signal value Sg₁, and the first blue signal value Sb₁ shown in FIG. 4 are output based on the reflected light L reflected off the skin of the face of the measurement operator. At the skin of the face, in-blood hemoglobin tends to reflect the red light RL at the skin, and the skin tends to absorb the green light GL and the blue light BL. The first red signal value Sr₁ is greater than the first green signal value Sg₁ and the first blue signal value Sb₁. The analog signal processing circuit 26 corrects the first red signal value Sr₁, the first green signal value Sg₁, and the first blue signal value Sb₁ by using the correction coefficients determined by using the color temperatures and the correction coefficient table.

The correction coefficient table is a data table that associates the color temperatures with the correction coefficients. The correction coefficient table is stored in the storage unit 50 in advance. The correction coefficients include a red signal correction coefficient kr, a green signal correction coefficient kg, and a blue signal correction coefficient kb. The red signal correction coefficient kr increases, decreases, or maintains the red signal value Sr. The red signal correction coefficient kr corresponds to an example of a first correction value. The green signal correction coefficient kg increases, decreases, or maintains the green signal value Sg. The green signal correction coefficient kg corresponds to an example of a second correction value. The blue signal correction coefficient kb is a coefficient that increases, decreases, or maintains the blue signal value Sb. The blue signal correction coefficient kb corresponds to an example of a third correction value. As an example, the analog signal processing circuit 26 adjusts the signal values by using Expressions (1), (2), and (3) below.

$$S {r}_{2} = kr \times {Sr}_{1} \qquad ( 1 )$$

$$S {g}_{2} = k g \times S {g}_{1} \qquad ( 2 )$$

$$S {b}_{2} = kb \times {Sb}_{1} \qquad ( 3 )$$

In Expressions (1), (2), and (3), the second red signal value Sr₂, the second green signal value Sg₂, and the second blue signal value Sb₂ indicate the red signal value Sr after the adjustment, the green signal value Sg after the adjustment, and the blue signal value Sb after the adjustment, respectively. When any of the correction coefficients is smaller than one, the signal value decreases. When any of the correction coefficients is greater than one, the signal value increases. When the correction coefficient is equal to one, the signal value does not change.

FIG. 5 shows an example of the signal values. FIG. 5 shows the red signal value Sr, the green signal value Sg, and the blue signal value Sb. FIG. 5 shows the second red signal value Sr₂, the second green signal value Sg₂, and the second blue signal value Sb₂ after the adjustment is performed on the first red signal value Sr₁, the first green signal value Sg₁, and the first blue signal value Sb₁.

The analog signal processing circuit 26 decreases the first red signal value Sr₁ to the second red signal value Sr₂ by using the red signal correction coefficient kr, as shown in FIG. 5. The analog signal processing circuit 26 causes the second red signal value Sr₂ to approach the output median MV by performing color temperature adjustment. The analog signal processing circuit 26 can suppress the influence of the color temperature of the outside light and the like on the red signal value Sr by performing the color temperature adjustment.

The analog signal processing circuit 26 may decrease the first green signal value Sg₁ to the second green signal value Sg₂ by using the green signal correction coefficient kg. The analog signal processing circuit 26 causes the second green signal value Sg₂ to approach the output median MV by performing the color temperature adjustment. The analog signal processing circuit 26 can suppress the influence of the color temperature of the outside light and the like on the green signal value Sg by performing the color temperature adjustment.

The analog signal processing circuit 26 may increase the first blue signal value Sb₁ to the second blue signal value Sb₂ by using the blue signal correction coefficient kb. The analog signal processing circuit 26 causes the second blue signal value Sb₂ to approach the output median MV by performing the color temperature adjustment. The analog signal processing circuit 26 can suppress the influence of the color temperature of the outside light and the like on the blue signal value Sb by performing the color temperature adjustment.

The second red signal value Sr₂, the second green signal value Sg₂, and the second blue signal value Sb₂ shown in FIG. 5 are adjusted so as to fall within an allowable range TR. The allowable range TR indicates a predetermined signal magnitude range having a median equal to the output median MV. It is preferable that the analog signal processing circuit 26 adjusts the second red signal value Sr₂, the second green signal value Sg₂, and the second blue signal value Sb₂ to values that fall within the allowable range TR by performing the adjustment. The accuracy of the detection of the pulse wave signal is improved by adjusting the second red signal value Sr₂, the second green signal value Sg₂, and the second blue signal value Sb₂ to values that fall within the allowable range TR.

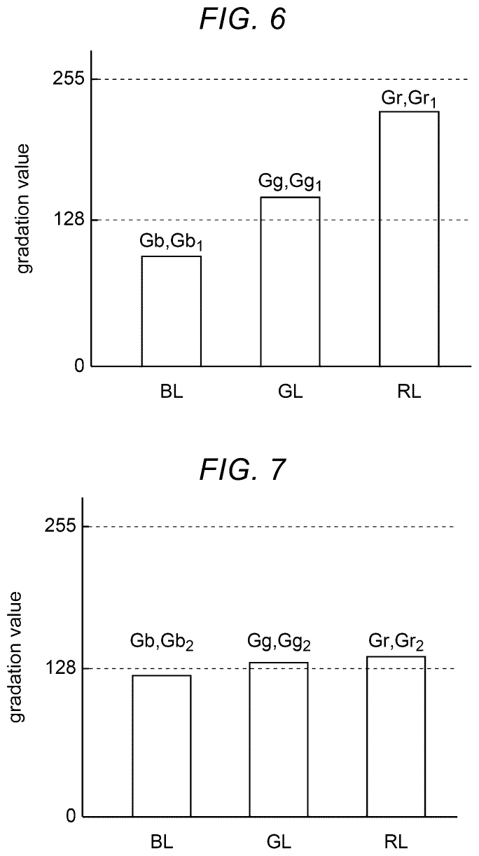

FIGS. 6 and 7 show examples of the grayscale values. FIGS. 6 and 7 show examples of the red grayscale value Gr, the green grayscale value Gg, and the blue grayscale value Gb. FIG. 6 shows the grayscale values as a result of the A/D conversion of the signal values shown in FIG. 4. FIG. 6 shows the grayscale values as a result of the A/D conversion of the signal values on which the adjustment has not been performed. FIG. 7 shows the grayscale values as a result of the A/D conversion of the signal values shown in FIG. 5. FIG. 7 shows the grayscale values as a result of the A/D conversion of the signal values on which the adjustment has been performed.

FIG. 6 shows a first red grayscale value Gr₁, a first green grayscale value Gg₁, and a first blue grayscale value Gb₁. The first red grayscale value Gr₁, the first green grayscale value Gg₁, and the first blue grayscale value Gb₁ are examples of the red grayscale value Gr, the green grayscale value Gg, and the blue grayscale value Gb, respectively. The first red grayscale value Gr₁, the first green grayscale value Gg₁, and the first blue grayscale value Gb₁ are values as a result of the A/D conversion of the first red signal value Sr₁, the first green signal value Sg₁, and the first blue signal value Sb₁, respectively. The first red grayscale value Gr₁ corresponding to the first red signal value Sr₁ on which the adjustment has not been performed is a value close to the upper limit of the grayscale range, as shown in FIG. 6. When the first red grayscale value Gr₁ is a value close to the upper limit of the grayscale range, the accuracy of the detection of the pulse wave signal may decrease.

FIG. 7 shows a second red grayscale value Gr₂, a second green grayscale value Gg₂, and a second blue grayscale value Gb₂. The second red grayscale value Gr₂, the second green grayscale value Gg₂, and the second blue grayscale value Gb₂ are examples of the red grayscale value Gr, the green grayscale value Gg, and the blue grayscale value Gb, respectively. The second red grayscale value Gr₂, the second green grayscale value Gg₂, and the second blue grayscale value Gb₂ are values as a result of the A/D conversion of the second red signal value Sr₂, the second green signal value Sg₂, and the second blue signal value Sb₂, respectively. The second red grayscale value Gr₂ corresponding to the second red signal value Sr₂ on which the adjustment has been performed is a value close to the median of the grayscale range, as shown in FIG. 7. When the second red grayscale value Gr₂ is a value close to the median of the grayscale range, the decrease in the accuracy of the detection of the pulse wave signal is suppressed.

The adjustment performed by the analog signal processing circuit 26 may be threshold adjustment using a predetermined output threshold TH. The output threshold TH corresponds to an example of a threshold. The output threshold TH is stored in the storage unit 50 in advance. The analog signal processing circuit 26 receives the electric signals relating to the image data acquired in the preparation step. The analog signal processing circuit 26 compares each of the signal values with the output threshold TH.

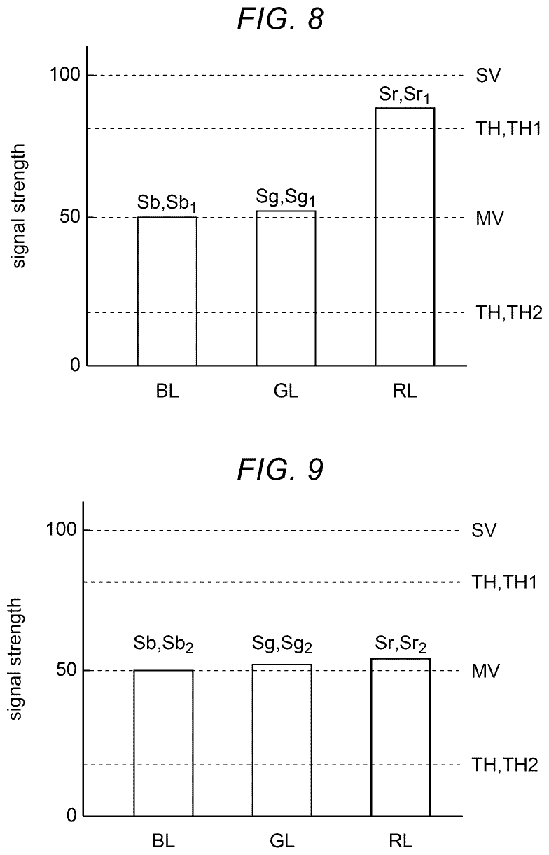

FIG. 8 shows an example of the signal values. FIG. 8 shows the red signal value Sr, the green signal value Sg, and the blue signal value Sb. FIG. 8 shows the first red signal value Sr₁, the first green signal value Sg₁, and the first blue signal value Sb₁ before the threshold adjustment is performed. FIG. 8 shows the saturated value SV and the output median MV characteristic of the imaging sensor 23. FIG. 8 shows an example of a first output threshold TH1 and a second output threshold TH2. The first output threshold TH1 and the second output threshold TH2 are examples of the output threshold TH.

The first output threshold TH1 indicates an upper limit. In a region where any of the output values is greater than the first output threshold TH1, the fluctuation of the grayscale value corresponding to the signal value decreases. In the region where the signal value is greater than the first output threshold TH1, the decrease in the fluctuation of the grayscale value decreases the accuracy of the detection of the pulse wave signal.

The second output threshold TH2 indicates a lower limit. In a region where any of the signal values is smaller than the second output threshold TH2, the fluctuation of the grayscale value corresponding to the signal value decreases. In the region where the signal value is smaller than the second output threshold TH2, a decrease in the fluctuation of the grayscale value decreases the accuracy of the detection of the pulse wave signal.

The analog signal processing circuit 26 compares each of the first red signal value Sr₁, the first green signal value Sg₁, and the first blue signal value Sb₁ shown in FIG. 8 with the output threshold TH. The analog signal processing circuit 26 determines that the first red signal value Sr₁ is greater than the first output threshold TH1. The analog signal processing circuit 26 performs signal processing that decreases the first red signal value Sr₁. The analog signal processing circuit 26 performs the signal processing that decreases the first red signal value Sr₁ to cause the adjusted first red signal value Sr₁ to approach the output median MV.

The first green signal value Sg₁ and the first blue signal value Sb₁ shown in FIG. 8 are greater than the second output threshold TH2. The analog signal processing circuit 26 does not perform signal processing that increases the first green signal value Sg₁ and the first blue signal value Sb₁. When any of the first red signal value Sr₁, the first green signal value Sg₁, and the first blue signal value Sb₁ is smaller than the second output threshold TH2, the analog signal processing circuit 26 performs the signal processing that increases the signal value smaller than the second output threshold TH2.

FIG. 9 shows an example of the signal values. FIG. 9 shows the red signal value Sr, the green signal value Sg, and the blue signal value Sb. FIG. 9 shows the second red signal value Sr₂, the second green signal value Sg₂, and the second blue signal value Sb₂ after the threshold adjustment is performed on the first red signal value Sr₁, the first green signal value Sg₁, and the first blue signal value Sb₁ shown in FIG. 8.

The second red signal value Sr₂ shown in FIG. 9 is smaller than the first red signal value Sr₁ shown in FIG. 8. The analog signal processing circuit 26 performs the threshold adjustment on the first red signal value Sr₁ to decrease the first red signal value Sr₁. The analog signal processing circuit 26 performs the threshold adjustment on the first red signal value Sr₁ to cause the first red signal value Sr₁ to approach the output median MV. The first red signal value Sr₁ is adjusted to the second red signal value Sr₂. The analog signal processing circuit 26 performs the threshold adjustment that causes the first red signal value Sr₁ to approach the output median MV, so that the measurement apparatus 10 can improve the accuracy of the detection of the red light RL.

The adjustment performed by the analog signal processing circuit 26 may be calculation adjustment using a calculation adjustment quantity generated by using the image data acquired in the preparation step. The image data corresponds to an example of captured data. The analog signal processing circuit 26 receives the electric signals relating to the image data acquired in the preparation step. The analog signal processing circuit 26 acquires the output median MV derived from the output resolution of the imaging sensor 23 by way of example. The analog signal processing circuit 26 may acquire a signal magnitude average that is the average of the signal values.

The analog signal processing circuit 26 calculates the calculation adjustment quantity by using the output median MV. The analog signal processing circuit 26 calculates a difference as a result of subtraction of the output median MV from the red signal value Sr as a red calculation adjustment quantity Cr. The red calculation adjustment quantity Cr corresponds to an example of a first adjustment quantity. The analog signal processing circuit 26 calculates a difference as a result of subtraction of the output median MV from the green signal value Sg as a green calculation adjustment quantity Cg. The green calculation adjustment quantity Cg corresponds to an example of a second adjustment quantity. The analog signal processing circuit 26 calculates a difference as a result of subtraction of the output median MV from the blue signal value Sb as a blue calculation adjustment quantity Cb. The analog signal processing circuit 26 adjusts the first red signal value Sr₁, the first green signal value Sg₁, and the first blue signal value Sb₁ to the second red signal value Sr₂, the second green signal value Sg₂, and the second blue signal value Sb₂ by using Expressions (4), (5), and (6) below by way of example.

$$S {r}_{2} = S {r}_{1} - Cr \qquad ( 4 )$$

$${Sg}_{2} = S {g}_{1} - Cg \qquad ( 5 )$$

$$S {b}_{2} = {Sb}_{1} - Cb \qquad ( 6 )$$

When any of the calculation adjustment quantities is a positive value, the signal value decreases. When any of the calculation adjustment quantities is a negative value, the signal value increases. When any of the calculation adjustment quantities is zero, the signal value does not change.

The calculation adjustment quantities may each be calculated by using the signal magnitude average. The signal magnitude average is used in place of the output median MV. The calculation adjustment quantities may each be generated by calculating the ratio between the signal value and the output median MV.

The adjustment performed by the analog signal processing circuit 26 may be comparison and adjustment that compares the signal values relating to the image data acquired in the preparation step and corrects the signal values based on the result of the comparison. The analog signal processing circuit 26 receives the electric signals relating to the image data acquired in the preparation step. The analog signal processing circuit 26 compares the red signal value Sr, the green signal value Sg, and the blue signal value Sb with each other.

As an example, the analog signal processing circuit 26 compares the first green signal value Sg₁ with the first red signal value Sr₁ and the first blue signal value Sb₁. The green light GL is light that allows the measurement apparatus to detect the pulse wave signal more readily than the red light RL and the blue light BL. The analog signal processing circuit 26 calculates the ratios of the first red signal value Sr₁ and the first blue signal value Sb₁ to the first green signal value Sg₁. The analog signal processing circuit 26 calculates a first signal magnitude ratio Sr/Sg and a second signal magnitude ratio Sb/Sg. The first signal magnitude ratio Sr/Sg is the ratio of the first red signal value Sr₁ to the first green signal value Sg₁. The second signal magnitude ratio Sb/Sg is the ratio of the first blue signal value Sb₁ to the first green signal value Sg₁.

The analog signal processing circuit 26 compares the first signal magnitude ratio Sr/Sg and the second signal magnitude ratio Sb/Sg with a standard ratio value. The standard ratio value is a predetermined value and is stored in the storage unit 50. The standard ratio value corresponds to an example of a ratio value. The standard ratio value includes a first standard ratio value that is an upper limit and a second standard ratio value that is a lower limit.

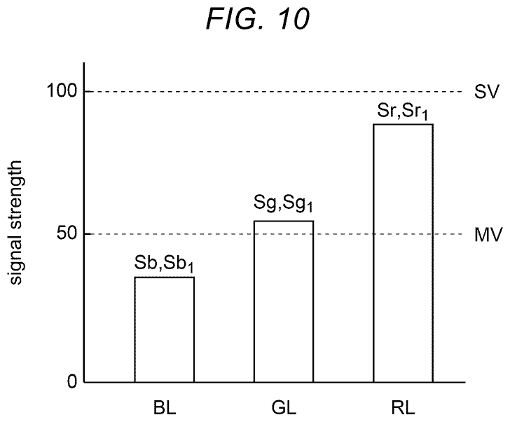

FIG. 10 shows an example of the signal values. FIG. 10 shows the red signal value Sr, the green signal value Sg, and the blue signal value Sb. FIG. 10 shows the first red signal value Sr₁, the first green signal value Sg₁, and the first blue signal value Sb₁ before the comparison and adjustment is performed. FIG. 10 shows the saturated value SV and the output median MV characteristic of the imaging sensor 23.

The first red signal value Sr₁ shown in FIG. 10 is greater than the first green signal value Sg₁. The analog signal processing circuit 26 compares the first signal magnitude ratio Sr/Sg with the first standard ratio value. When determining that the first signal magnitude ratio Sr/Sg is greater than the first standard ratio value, the analog signal processing circuit 26 performs signal processing on the first red signal value Sr₁. The analog signal processing circuit 26 performs the signal processing that decreases the first red signal value Sr₁. The analog signal processing circuit 26 adjusts the first red signal value Sr₁ to the second red signal value Sr₂ close to the output median MV. When determining that the first signal magnitude ratio Sr/Sg is smaller than the first standard ratio value, the analog signal processing circuit 26 does not perform the signal processing on the first red signal value Sr₁.

The first blue signal value Sb₁ shown in FIG. 10 is smaller than the first green signal value Sg₁. The analog signal processing circuit 26 compares the second signal magnitude ratio Sb/Sg with the second standard ratio value. When determining that the second signal magnitude ratio Sb/Sg is smaller than the second standard ratio value, the analog signal processing circuit 26 performs signal processing on the first blue signal value Sb₁. The analog signal processing circuit 26 performs signal processing that increases the first blue signal value Sb₁. The analog signal processing circuit 26 adjusts the first blue signal value Sb₁ to the second blue signal value Sb₂ close to the output median MV. When determining that the second signal magnitude ratio Sb/Sg is greater than the second standard ratio value, the analog signal processing circuit 26 does not perform the signal processing on the first blue signal value Sb₁.

The digital signal processing circuit 27 shown in FIG. 3 receives the grayscale values output from the analog signal processing circuit 26. The grayscale values include the red grayscale value Gr, the green grayscale value Gg, and the blue grayscale value Gb. The digital signal processing circuit 27 performs correction such as pixel interpolation, γ correction, color correction, contour coordination, and noise reduction on the grayscale values. The digital signal processing circuit 27 outputs video image data containing the corrected grayscale values to the control unit 40.

The imaging unit 20 may output the red signal value Sr, the green signal value Sg, and the blue signal value Sb, which are analog signals, to the control unit 40 in the form of video image data containing the three signal values. The control unit 40 acquires the red signal value Sr, the green signal value Sg, and the blue signal value Sb.

The display unit 30 displays various images under the control of the control unit 40. The display unit 30 receives display data from the control unit 40 and displays various images based on the display data. The display unit 30 may display video images captured by the imaging unit 20 based on the video image data. The display unit 30 may display the captured image 100 based on image data contained in the video image data.

The display unit 30 transmits the input signals to the control unit 40. The display unit 30 transmits the input signals to the control unit 40 to cause the control unit 40 to perform various types of control. As an example, the display unit 30 transmits a display instruction signal used to display living body information as one of the input signals to the control unit 40. The control unit 40 generates living body information display data that causes the display unit 30 to display living body information based on the display instruction signal. The control unit 40 transmits the living body information display data to the display unit 30. The display unit 30 displays a screen containing the living body information based on the living body information display data.

The control unit 40 is a controller that controls operations of the various units. The control unit 40 is a processor including a central processing unit (CPU) by way of example. The control unit 40 is configured with one or more processors. The control unit 40 is communicatively connected to the imaging unit 20, the display unit 30, and the like. The control unit 40 functions as a region setting portion 41, an analysis portion 43, and a display control portion 45 by executing the living body analysis program PG. The control unit 40 may function as a signal processing portion 47 by executing the living body analysis program PG. The control unit 40 may function as a functional portion other than the region setting portion 41, the analysis portion 43, the display control portion 45, and the signal processing portion 47 by executing the living body analysis program PG. The control unit 40 corresponds to an example of a computer.

The region setting portion 41 acquires the video image data transmitted from the imaging unit 20. The region setting portion 41 acquires the multiple sets of image data contained in the video image data. The region setting portion 41 performs face recognition for each of the image data sets. The face recognition is processing that extracts a face image region 121 by detecting feature points contained in the image data and matching the feature points against a face image database registered in advance. The feature points contained in the image data are, for example, the positions and contours of the eyes, nose, and mouth. The face image database is stored in the storage unit 50 in advance. When the measurement apparatus 10 is connected to a server via a network, the face image database may be stored in the server in advance. The measurement apparatus 10 acquires the face image database from the server. The face image region 121 is a region where the face of the measurement operator is displayed. The region setting portion 41 identifies the face image region 121 by performing the face recognition.

After identifying the face image region 121, the region setting portion 41 determines a detection region 201. As an example, the region setting portion 41 determines the detection region 201 based on set region information SI, which will be described later. The region setting portion 41 may determine the detection region 201 by using the video image data output from the imaging unit 20.

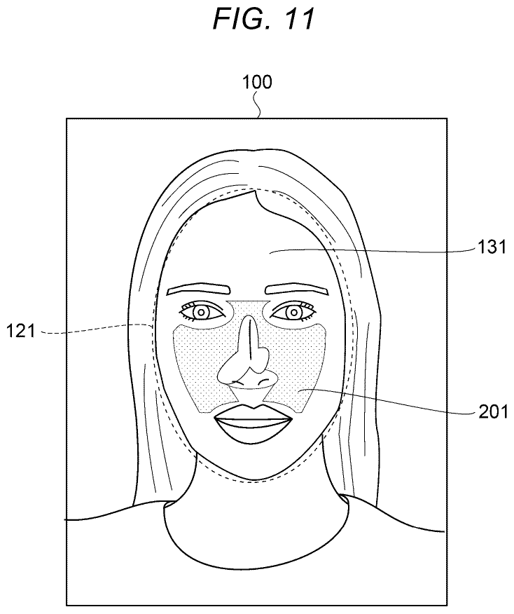

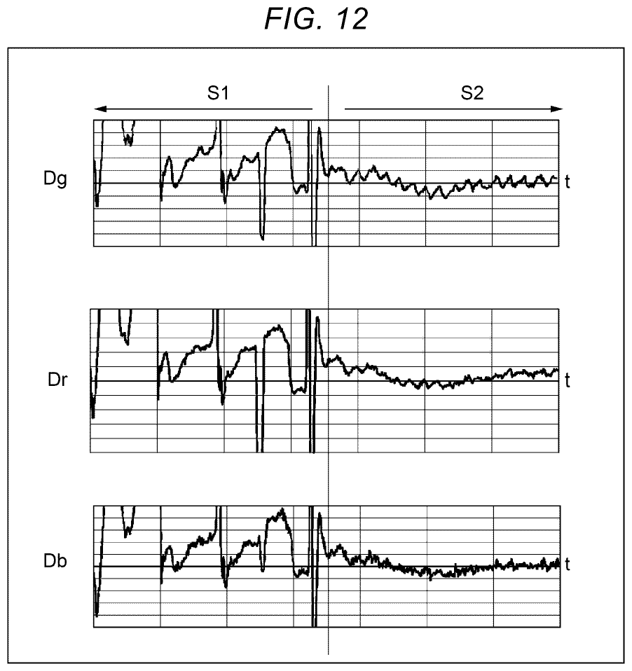

FIG. 11 shows an example of the detection region 201. The detection region 201 is determined by the region setting portion 41 excluding a non-detection region 131 from the face image region 121. The region setting portion 41 determines a portion of the face image region 121 as the detection region 201. FIG. 11 shows the face image region 121, the detection region 201, and the non-detection region 131 superimposed on the captured image 100. The captured image 100 is an example of an image contained in the video image captured by the imaging unit 20.

The detection region 201 is determined by excluding the non-detection region 131 from the face image region 121. The non-detection region 131 includes a region that is readily affected by noise such as a body motion, a region in which the pulse wave signal is hardly detected, and the like. The measurement apparatus 10 detects the pulse wave signal by using the detection region 201, which is the face image region 121 from which the non-detection region 131 is excluded. The measurement apparatus 10 can improve the accuracy of the detection of the pulse wave signal by using the detection region 201 to detect the pulse wave signal.

The analysis portion 43 shown in FIG. 3 calculates the pulse wave signal by using the red grayscale value Gr, the green grayscale value Gg, and the blue grayscale value Gb. The red grayscale value Gr, the green grayscale value Gg, and the blue grayscale value Gb are generated by performing the A/D conversion on the red signal value Sr, the green signal value Sg, and the blue signal value Sb, respectively. The analysis portion 43 calculates the pulse wave signal based on the red signal value Sr, the green signal value Sg, and the blue signal value Sb. The analysis portion 43 calculates the living body information based on the pulse wave signal. The analysis portion 43 acquires the multiple sets of image data contained in the video image data from the imaging unit 20. The analysis portion 43 acquires the detection region 201 from the region setting portion 41. The analysis portion 43 detects the grayscale values in the detection region 201 from each of the image data sets, and detects the pulse wave signal of the measurement operator M based on the grayscale values. The analysis portion 43 identifies multiple pixels corresponding to the detection region 201. The analysis portion 43 detects the grayscale value of each of the identified multiple pixels. The analysis portion 43 generates a detected grayscale value by using the multiple grayscale values in the detection region 201. The detected grayscale value is, for example, the average of the grayscale values of the pixels in the detection region 201. The detected grayscale value is calculated for each of the image data sets in the video image data. The analysis portion 43 detects the pulse wave signal of the measurement operator M by using the detected grayscale values. The analysis portion 43 corresponds to an example of a calculation portion.

The analysis portion 43 detects the pulse wave signal by using the detected grayscale values. As an example, the analysis portion 43 detects the pulse wave signal by using at least one of a red detected grayscale value Dr, a green detected grayscale value Dg, and a blue detected grayscale value Db. The red detected grayscale value Dr is calculated by using the red grayscale values Gr as a result of the A/D conversion of the red signal values Sr output from the pixels corresponding to the detection region 201. The green detected grayscale value Dg is calculated by using the green grayscale values Gg as a result of the A/D conversion of the green signal values Sg output from the pixels corresponding to the detection region 201. The blue detected grayscale value Db is calculated by using the blue grayscale values Gb as a result of the A/D conversion of the blue signal values Sb output from the pixels corresponding to the detection region 201. The pulse wave signal is detected based on the green detected grayscale value Dg. The pulse wave signal is detected based on the difference between the green detected grayscale value Dg and at least one of the red detected grayscale value Dr and the blue detected grayscale value Db.

FIG. 12 shows an example of the detected grayscale values. FIG. 12 shows the red detected grayscale value Dr, the green detected grayscale value Dg, and the blue detected grayscale value Db. FIG. 12 shows temporal changes in the red detected grayscale value Dr, the green detected grayscale value Dg, and the blue detected grayscale value Db in the form of waveform signals. FIG. 12 shows the red detected grayscale value Dr, the green detected grayscale value Dg, and the blue detected grayscale value Db in a body motion segment S1, and the red detected grayscale value Dr, the green detected grayscale value Dg, and the blue detected grayscale value Db in a rest segment S2. The body motion segment S1 is a segment in which a face motion or a facial expression changes. The rest segment S2 is a segment in which a change in face motion or facial expression is smaller than the amount of a predetermined change.

The green detected grayscale value Dg fluctuates due to the influence of a body motion in the body motion segment S1. In the body motion segment S1, it is difficult to detect the pulse wave signal contained in the green detected grayscale value Dg due to fluctuating noise. In the rest segment S2, the influence of the fluctuating noise due to a body motion decreases, so that the analysis portion 43 can detect the pulse wave signal by using the green detected grayscale value Dg.

The red detected grayscale value Dr fluctuates due to the influence of a body motion in the body motion segment S1. In the body motion segment S1, it is difficult to detect the pulse wave signal contained in the red detected grayscale value Dr due to fluctuating noise. In the rest segment S2, the influence of fluctuating noise due to a body motion decreases, but the SN ratio of the red detected grayscale value Dr is small, so that it is difficult for the analysis portion 43 to detect the pulse wave signal.

The blue detected grayscale value Db fluctuates due to the influence of a body motion in the body motion segment S1. In the body motion segment S1, it is difficult to detect the pulse wave signal contained in the blue detected grayscale value Db due to fluctuating noise. In the rest segment S2, the influence of fluctuating noise due to a body motion decreases, but the SN ratio of the blue detected grayscale value Db is small, so that it is difficult for the analysis portion 43 to detect the pulse wave signal.

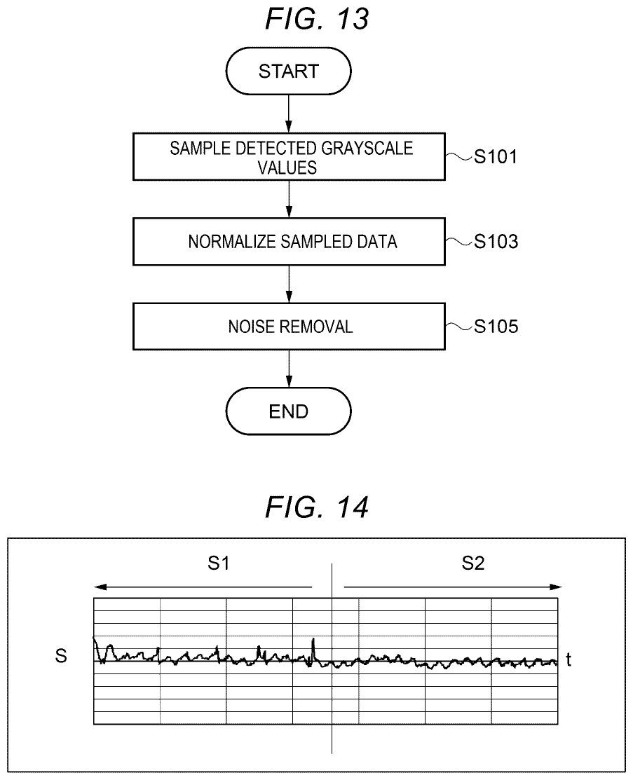

The analysis portion 43 detects the pulse wave signal by using the red detected grayscale value Dr, the green detected grayscale value Dg, and the blue detected grayscale value Db shown in FIG. 12. As an example, the analysis portion 43 detects the pulse wave signal by the analysis procedure shown in FIG. 13.

FIG. 13 shows an example of the analysis procedure for detecting the pulse wave signal. FIG. 13 is a flowchart showing the example of the analysis procedure. The analysis procedure shown in FIG. 13 is executed by the analysis portion 43. In the analysis procedure shown in FIG. 13, the pulse wave signal is detected by using the red detected grayscale value Dr, the green detected grayscale value Dg, and the blue detected grayscale value Db.

In step S101, the analysis portion 43 performs sampling of each of the detected grayscale values at predetermined time intervals. The time interval and the sampling frequency are set as appropriate. The time interval is preferably a period containing one or more pulse waves. The time interval ranges, for example, from 3 to 10 seconds. The sampling frequency is, for example, higher than or equal to 10 Hz but lower than or equal to 50 Hz. The analysis portion 43 acquires sampled data by performing the sampling. The sampled data contains the sampled red detected grayscale values Dr, green detected grayscale values Dg, and blue detected grayscale values Db.

After performing the sampling, the analysis portion 43 normalizes the sampled data in step S103. The analysis portion 43 normalizes the multiple green detected grayscale values Dg contained in the sampled data.

The analysis portion 43 calculates a green average Gmean, which is the average of the multiple green detected grayscale values Dg, and a green standard deviation Gstd, which is the standard deviation of the multiple green detected grayscale values Dg. The analysis portion 43 normalizes each of the green detected grayscale values Dg by using Expression (7) below.

$$G n o r {m}_{n} = ( Gn - Gmean ) / Gstd \qquad ( 7 )$$

In Expression (7), n is any integer greater than or equal to one. Gn is the n-th green detected grayscale value Dg. Gnormₙ is a value as a result of the normalization of the n-th green detected grayscale value Dg.

The analysis portion 43 normalizes the multiple red detected grayscale values Dr and the multiple blue detected grayscale values Db contained in the sampled data, as the green detected grayscale values Dg. The analysis portion 43 calculates a red average Rmean, which is the average of the multiple red detected grayscale values Dr, and a red standard deviation Rstd, which is the standard deviation of the multiple red detected grayscale values Dr. The analysis portion 43 calculates a blue average Bmean, which is the average of the multiple blue detected grayscale values Db, and a blue standard deviation Bstd, which is the standard deviation of the multiple blue detected grayscale values Db. The analysis portion 43 normalizes each of the red detected grayscale values Dr and each of the blue detected grayscale values Db by using Expressions (8) and (9) below.

$$R n o r {m}_{n} = ( Rn - Rmean ) / Rstd \qquad ( 8 )$$

$$Rnor {m}_{n} = ( Bn - Bmean ) / Bstd \qquad ( 9 )$$

In Expressions (8) and (9), n is any integer greater than or equal to one. Rn is the n-th red detected grayscale value Dr. Rnormₙ is a value as a result of the normalization of the n-th red detected grayscale value Dr. Bn is the n-th blue detected grayscale value Db. Bnormₙ is a value as a result of the normalization of the n-th blue detected grayscale value Db.

After normalizing the sampled data, the analysis portion 43 performs noise removal in step S105. The analysis portion 43 performs the noise removal by using the normalized green detected grayscale values Dg, the normalized red detected grayscale values Dr, and the normalized blue detected grayscale values Db. The analysis portion 43 performs the noise removal by using Expression (10) below to generate a noise-removed signal S:

$$Sn = {Gnorm}_{n} + α {Bnorm}_{n} + β Rnor {m}_{n} \qquad ( 10 )$$

In Expression (10), n is any integer greater than or equal to one. Sn is the n-th noise-removed signal S. α is a first coefficient, and β is a second coefficient.

As an example, α and β are each −0.5. When α and β are negative values, the analysis portion 43 detects the noise-removed signal S by subtracting the normalized red detected grayscale values Dr and the normalized blue detected grayscale values Db from the normalized green detected grayscale values Dg. At least one of α and β may be zero. When α=0 and β=−1, the analysis portion 43 detects the noise-removed signal S by calculating the difference between the green detected grayscale values Dg and the red detected grayscale values Dr. When α=−1 and β=0, the analysis portion 43 detects the noise-removed signal S by calculating the difference between the green detected grayscale values Dg and the blue detected grayscale values Db. The coefficients α and β are set as appropriate in accordance with the state of the noise removal.

FIG. 14 shows a result of the analysis of the noise-removed signal S. FIG. 14 shows the noise-removed signal S analyzed based on the red detected grayscale value Dr, the green detected grayscale value Dg, and the blue detected grayscale value Db shown in FIG. 12. FIG. 14 shows the noise-removed signal S detected when α=−0.5 and β=−0.5 are substituted into Expression (10). FIG. 14 shows the noise-removed signal S in the body motion segment S1 and the rest segment S2.

The noise-removed signal S corresponds to the pulse wave signal, as shown in FIG. 14. Noise components such as the body motion have been removed from the noise-removed signal S. The analysis portion 43 detects the noise-removed signal S as the pulse wave signal. The noise-removed signal S in the rest segment S2 has a clearer signal waveform than the green detected grayscale value Dg. The noise-removed signal S in the body motion segment S1 is adjusted to a signal waveform corresponding to the pulse wave signal. Performing the noise removal allows the analysis portion 43 to detect the pulse wave signals in the body motion segment S1 and the rest segment S2.

The analysis portion 43 calculates living body information such as the pulse wave by using the noise-removed signal S. The analysis portion 43 acquires the noise-removed signal S as the pulse wave signal. The analysis portion 43 calculates living body information such as the pulse by calculating the cycle, the amplitude, and the like of the pulse wave signal. The analysis portion 43 transmits the living body information including the pulse wave signal to the display control portion 45. The analysis portion 43 may store the living body information and the like in the storage unit 50.

The display control portion 45 shown in FIG. 3 controls display operation performed by the display unit 30. The display control portion 45 acquires the living body information including the pulse wave signal from the analysis portion 43. The display control portion 45 generates the living body information display data used to display the living body information. The display control portion 45 transmits the living body information display data to the display unit 30. The display control portion 45 causes the display unit 30 to display the living body information display data. The display control portion 45 can notify the measurement operator of the result of the detection of the living body information by causing the display unit 30 to display the living body information display data.

The display control portion 45 may generate message data indicating the state of the operation of the living body analysis program PG. The message data include a start message, an execution message, an end message, and other messages. The start message indicates that the detection of the living body information is started. The execution message indicates that the living body information is being detected. The end message indicates that the detection of the living body information has ended. The display control portion 45 transmits the message data to the display unit 30. The display control portion 45 causes the display unit 30 to display the message data.

When the control unit 40 receives the signal values from the imaging unit 20, the signal processing portion 47 performs the adjustment of the signal values. The signal processing portion 47 performs the A/D conversion on the signal values on which the adjustment has been performed to convert the signal values into grayscale values. The signal processing portion 47 functions as the analog signal processing circuit 26 and the digital signal processing circuit 27. The signal processing portion 47 may acquire the detection region 201 from the region setting portion 41 and perform the adjustment by using the detection region 201.

The storage unit 50 stores various programs, various data, and the like. The storage unit 50 stores the living body analysis program PG and the set region information SI. The storage unit 50 stores a document creation program, a spreadsheet program, and the like. The storage unit 50 may store various data such as video image data and living body information. The storage unit 50 may store the correction coefficient table, the output threshold TH, and the like. The correction coefficient table contains the red signal correction coefficient kr, the green signal correction coefficient kg, and the blue signal correction coefficient kb. The storage unit 50 may store the face image database. The storage unit 50 is configured with semiconductor memories such as a RAM (random access memory) and a ROM (read only memory). The storage unit 50 may function as a work area used by the control unit 40. The storage unit 50 corresponds to an example of a storage portion.

The living body analysis program PG is a program that causes the measurement apparatus 10 to detect living body information including the pulse wave signal. The living body analysis program PG is executed by the control unit 40. When the living body analysis program PG is executed by the control unit 40, the control unit 40 functions as the various functional portions. The living body analysis program PG detects various pieces of living body information based on the pulse wave signal. The living body analysis program PG may be executed in the background when the control unit 40 executes the document creation program or the like. The living body analysis program PG corresponds to an example of a living body information acquisition program.

The set region information SI is information on the non-detection region 131. The non-detection region 131 is used when the control unit 40 determines the detection region 201. The non-detection region 131 falls within the face image region 121. The control unit 40 determines the detection region 201 by excluding the non-detection region 131 from the face image region 121. The non-detection region 131 includes one or more site regions. As an example, the set region information SI is information indicating that the one or more site regions form the non-detection region 131. The site regions are a peripheral region, a head region, a head hair region, an orbit region, a nasal cavity region, a lip region, a forehead region, a lower jaw region, and the like. The peripheral region is a region including peripheral sites such as the contour of the face or a boundary with the hair. The head region is a region corresponding to the head. The head hair region is a region including the head hair. The orbit region is a region corresponding to the orbits. The nasal cavity region is a region corresponding to the nasal cavity. The lip region is a region corresponding to the lip. The forehead region is a region corresponding to the forehead. The lower jaw region is a region corresponding to the lower jaw.

The measurement apparatus 10 includes the imaging sensor 23, which captures the reflected light L reflected off the measurement operator and containing the red light RL having the red wavelength and the green light GL having the green wavelength different from the red wavelength, and outputs the red signal value Sr relating to the red light RL and the green signal value Sg relating to the green light GL, the analog signal processing circuit 26, which performs the adjustment on the red signal value Sr, which causes the red signal value Sr to approach the output median MV derived from the output resolution of the imaging sensor 23, and the analysis portion 43, which calculates living body information based on the red signal value Sr and the green signal value Sg.

The measurement apparatus 10 can improve the accuracy of the detection of the pulse wave signal by performing the adjustment on the red signal value Sr or the like.

When the red signal value Sr is greater than the predetermined output threshold TH, the analog signal processing circuit 26 preferably decreases the red signal value Sr.

The measurement apparatus 10 can suppress a decrease in the accuracy of the detection using the red light RL by decreasing the red signal value Sr.

When the ratio of the red signal value Sr to the green signal value Sg is greater than a predetermined standard ratio value, the analog signal processing circuit 26 preferably decreases the red signal value Sr.

The measurement apparatus 10 can suppress the decrease in the accuracy of the detection using the red light RL by decreasing the red signal value Sr.

The analog signal processing circuit 26 preferably performs the adjustment on the green signal value Sg.

The measurement apparatus 10 can improve the accuracy of the detection of the pulse wave signal by performing the adjustment on the green signal value Sg.

As the adjustment, the analog signal processing circuit 26 preferably adjusts the red signal value Sr and the green signal value Sg to the output median MV derived from the output resolution.

The measurement apparatus 10 can improve the accuracy of the detection of the pulse wave signal by adjusting the red signal value Sr and the green signal value Sg to the output median MV derived from the output resolution of the imaging sensor 23.

The analog signal processing circuit 26 preferably performs the adjustment on the red signal value Sr and the green signal value Sg by adjusting the signal amplification factor.

The measurement apparatus 10 can readily adjust the red signal value Sr and the green signal value Sg.

The measurement apparatus 10 includes the storage unit 50, which stores the red signal correction coefficient kr used to correct the red signal value Sr and the green signal correction coefficient kg used to correct the green signal value Sg. The analog signal processing circuit 26 preferably performs the adjustment on the red signal value Sr and the green signal value Sg by using the red signal correction coefficient kr and the green signal correction coefficient kg.

The measurement apparatus 10 can readily adjust the red signal value Sr and the green signal value Sg.

The imaging unit 20 captures the reflected light L to generate image data. The analog signal processing circuit 26 preferably calculates the red calculation adjustment quantity Cr used to adjust the red signal value Sr and the green calculation adjustment quantity Cg used to adjust the green signal value Sg based on the image data, and performs the adjustment on the red signal value Sr and the green signal value Sg by using the red calculation adjustment quantity Cr and the green calculation adjustment quantity Cg.

The measurement apparatus 10 can readily adjust the red signal value Sr and the green signal value Sg.

The imaging unit 20 outputs the blue signal value Sb relating to the blue light BL having the blue wavelength different from the red wavelength and the green wavelength. The analog signal processing circuit 26 preferably performs the adjustment on the blue signal value Sb.

The measurement apparatus 10 can improve the accuracy of the detection of the pulse wave signal.

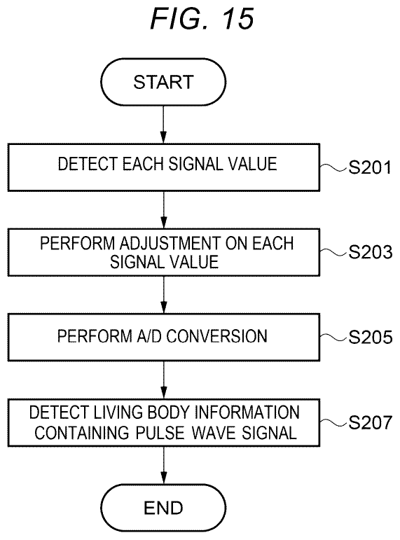

FIG. 15 shows an example of the control procedure executed by the measurement apparatus 10. FIG. 15 shows the control procedure of receiving the signal values and detecting living body information including the pulse wave signal by using the signal values. The control procedure is executed by the control unit 40 executing the living body analysis program PG. FIG. 15 is a flowchart showing the control procedure.

In step S201, the measurement apparatus 10 detects the signal values. The imaging unit 20 of the measurement apparatus 10 captures the reflected light L reflected off the face or the like of the measurement operator. The imaging unit 20 generates video image data by capturing the reflected light L. The video image data contains multiple sets of image data. The imaging unit 20 detects the signal values that constitute the image data sets. The analog signal processing circuit 26 of the imaging unit 20 detects, as the signal values, the red signal value Sr relating to the red light RL having the red wavelength, the green signal value Sg relating to the green light GL having the green wavelength, and the blue signal value Sb relating to the blue light BL having the blue wavelength.

After detecting the signal values, the measurement apparatus 10 performs the adjustment on the signal values in step S203. As an example, the analog signal processing circuit 26 increases or decreases each of the signal values by adjusting the signal amplification factor of the signal value. The analog signal processing circuit 26 causes each of the signal values to approach the output median MV derived from the output resolution of the imaging sensor 23 provided in the imaging unit 20 by increasing or decreasing the signal value. The analog signal processing circuit 26 performs as the adjustment at least one of the color temperature adjustment, the threshold adjustment, the calculation adjustment, and the comparison and adjustment.

After performing the adjustment on each of the signal values, the measurement apparatus 10 performs the A/D conversion on the signal value in step S205. The analog signal processing circuit 26 performs the A/D conversion on the red signal value Sr, the green signal value Sg, and the blue signal value Sb to convert the signal values into the red grayscale value Gr, the green grayscale value Gg, and the blue grayscale value Gb, respectively. The digital signal processing circuit 27 performs various types of digital correction on the red grayscale value Gr, the green grayscale value Gg, and the blue grayscale value Gb. After the digital signal processing circuit 27 performs the digital correction on each of the gradation values, the imaging unit 20 outputs video image data containing image data configured with the red grayscale value Gr, the green grayscale value Gg, and the blue grayscale value Gb to the control unit 40.

After performing the A/D conversion on each of the signal values, the measurement apparatus 10 detects living body information including the pulse wave signal in step S207. The analysis portion 43 of the control unit 40 acquires the video image data and the detection region 201 determined by the region setting portion 41. The analysis portion 43 acquires the red detected grayscale values Dr, the green detected grayscale values Dg, and the blue detected grayscale values Db in the detection region 201 by using the image data contained in the video image data and the detection region 201. The analysis portion 43 detects the pulse wave signal based on the red detected grayscale values Dr, the green detected grayscale values Dg, and the blue detected grayscale values Db. The analysis portion 43 detects living body information by using the pulse wave signal.

The living body analysis program PG causes the control unit 40 coupled to the imaging unit 20 including the imaging sensor 23, which captures the reflected light L reflected off the measurement operator and containing the red light RL having the red wavelength and the green light GL having the green wavelength different from the red wavelength, and outputs the red signal value Sr relating to the red light RL and the green signal value Sg relating to the green light GL, to calculate living body information based on the red signal value Sr and the green signal value Sg on which the adjustment, which causes two signal values to approach the output median MV derived from the output resolution of the imaging sensor 23, has been performed.

The measurement apparatus 10 can improve the accuracy of the detection of the pulse wave signal by performing the adjustment on the red signal value Sr or the like.

The measurement apparatus 10 shown in FIG. 3 uses the analog signal processing circuit 26 to perform the adjustment that causes each of the signal values to approach the output median MV derived from the output resolution of the imaging sensor 23. The measurement apparatus 10 may perform processing corresponding to the adjustment by using the color filter 24 having a changed configuration. The measurement apparatus 10 may include a color filter dedicated for living body information measurement in place of the color filter 24. The dedicated color filter is an example of the color filter 24. As an example, the dedicated color filter is designed to have a configuration in which the light transmittance for the red light RL is lower than the light transmittance for the green light GL and the light transmittance for the blue light BL. In the reflected light L reflected off the measurement operator, the amount of the red light RL is greater than the amount of the green light GL and the amount of the blue light BL. Providing the dedicated color filter, which has light transmittance for the red light RL lower than the light transmittance for the green light GL and the light transmittance for the blue light BL, causes the red signal value Sr relating to the red light RL to approach the output median MV derived from the output resolution of the imaging sensor 23. Since the red signal value Sr approaches the output median MV derived from the output resolution of the imaging sensor 23, the measurement apparatus 10 can suppress a decrease in the accuracy of the measurement of the pulse wave signal.

The dedicated color filter may have a configuration in which the light transmittance thereof is so adjusted that the signal values each coincide or substantially coincide with the output median MV of the imaging sensor 23. The state in which the signal values each substantially coincide with the output median MV of the imaging sensor 23 indicates that the signal value falls within the predetermined allowable range TR. The light transmittance for the red light RL is set within a range over which the red signal value Sr coincides or substantially coincides with the output median MV of the imaging sensor 23. The light transmittance for the green light GL is set within a range over which the green signal value Sg coincides or substantially coincides with the output median MV of the imaging sensor 23. The light transmittance for the blue light BL is set within a range over which the blue signal value Sb coincides or substantially coincides with the output median MV of the imaging sensor 23.

The dedicated color filter has a first portion that transmits the red light RL, a second portion that transmits the green light GL, and a third portion that transmits the blue light BL, as the color filter 24. The dedicated color filter may have a configuration in which the first portion is smaller than the second portion and the third portion. When the first portion is smaller than the second portion and the third portion, the red signal value Sr decreases. The red signal value Sr approaches the output median MV derived from the output resolution of the imaging sensor 23. Since the red signal value Sr approaches the output median MV derived from the output resolution of the imaging sensor 23, the measurement apparatus 10 can suppress a decrease in the accuracy of the measurement of the pulse wave signal.

The measurement apparatus 10 includes the dedicated color filter, which transmits reflected light L reflected off the measurement operator and containing the red light RL having the red wavelength and the green light GL having the green wavelength different from the red wavelength, the imaging sensor 23, which captures the reflected light L having passed through the dedicated color filter and outputs the red signal value Sr relating to the red light RL and the green signal value Sg relating to the green light GL, and the analog signal processing circuit 26, which calculates living body information based on the red signal value Sr and the green signal value Sg. The dedicated color filter has light transmittance that causes the red signal value Sr to approach the output median MV derived from the output resolution of the imaging sensor 23.

The measurement apparatus 10 can improve the accuracy of the measurement of the pulse wave signal.

What is claimed is:

1. A living body information acquisition apparatus comprising:
an imaging portion configured to capture reflected light reflected off a living body and containing first wavelength light having a first wavelength and second wavelength light having a second wavelength different from the first wavelength, and output a first output value relating to the first wavelength light and a second output value relating to the second wavelength light;
a data processing portion configured to perform data correction on the first output value to cause the first output value to approach a median derived from an output resolution of the imaging portion; and
a calculation portion configured to calculate living body information based on the first output value and the second output value.

2. The living body information acquisition apparatus according to claim 1, wherein
when the first output value is greater than a predetermined threshold, the data processing portion is configured to decrease the first output value.

3. The living body information acquisition apparatus according to claim 1, wherein
when a ratio of the first output value to the second output value is greater than a predetermined ratio value, the data processing portion is configured to decrease the first output value.

4. The living body information acquisition apparatus according to claim 1, wherein
the data processing portion is configured to perform the data correction on the second output value.

5. The living body information acquisition apparatus according to claim 4, wherein
the data processing portion is configured to adjust the first output value and the second output value to the median derived from the output resolution as the data correction.

6. The living body information acquisition apparatus according to claim 4, wherein
the data processing portion is configured to perform the data correction on the first output value and the second output value by adjusting a signal amplification factor.

7. The living body information acquisition apparatus according to claim 4, further comprising
a storage portion configured to store a first correction value used to correct the first output value and a second correction value used to correct the second output value,
wherein the data processing portion is configured to perform the data correction on the first output value and the second output value by using the first correction value and the second correction value.

8. The living body information acquisition apparatus according to claim 4, wherein
the imaging portion is configured to capture the reflected light to generate captured data, and
the data processing portion is configured to
calculate a first adjustment quantity used to adjust the first output value and a second adjustment quantity used to adjust the second output value based on the captured data, and
perform the data correction on the first output value and the second output value by using the first adjustment quantity and the second adjustment quantity.

9. The living body information acquisition apparatus according to claim 4, wherein
the imaging portion is configured to output a third output value relating to third wavelength light having a third wavelength different from the first wavelength and the second wavelength, and
the data processing portion is configured to perform the data correction on the third output value.

10. A living body information acquisition apparatus comprising:
a color filter configured to transmit reflected light reflected off a living body and containing first wavelength light having a first wavelength and second wavelength light having a second wavelength different from the first wavelength;
an imaging portion configured to capture the reflected light passing through the color filter and output a first output value relating to the first wavelength light and a second output value relating to the second wavelength light; and
a calculation portion configured to calculate living body information based on the first output value and the second output value,
wherein the color filter has light transmittance that causes the first output value to approach a median derived from an output resolution of the imaging portion.

11. A non-transitory computer-readable storage medium storing a living body information acquisition program configured to cause a computer coupled to an imaging unit including an imaging portion configured to capture reflected light reflected off a living body and containing first wavelength light having a first wavelength and second wavelength light having a second wavelength different from the first wavelength and output a first output value relating to the first wavelength light and a second output value relating to the second wavelength light to
calculate living body information based on the first output value and the second output value on which data correction that causes the first output value and the second output value to approach a median derived from an output resolution of the imaging portion is performed.
